(19) United States
(12) Patent Application Publication
(10) Pub. No.: US 2003/0068385 A1
(43) Pub. Date: Apr. 10, 2003

(54) Accelerated action fatty acid (AAFA) promotes health of normal tissues and minimizes the toxic side effects of chemotherapy

(76) Inventors: Mary Pat Moyer, San Antonio, TX (US); W. Elaine Hardman, Baton Rouge, LA (US); Ivan L. Cameron, San Antonio, TX (US)

Correspondence Address: Mary Pat Moyer, Suite B-200, 12000 Network Blvd., San Antonio, TX, 78249, US

(21) Appl. No.: 10/102,907

(22) Filed: Mar. 22, 2002

Related U.S. Application Data
(60) Provisional application No. 60/278,138, filed on Mar. 23, 2001.

(51) Int. Cl. A61K 35/70; A61K 35/72; A61K 31/22; A61K 35/78

(52) U.S. Cl. 424/523; 424/725; 424/195.16; 424/195.17; 514/549

(57) ABSTRACT

The invention is a formulation and method of use for a nutritional dietary supplement to prevent the side effects of cancer therapy and thus augment the efficacy of treatment. Cancer therapy refers to treatment with various formulations of chemicals (chemotherapy), or combined modality therapies that may include chemotherapy, radiation therapy, and/or surgery. The formulation of the nutritional supplement is highly enriched in .omega.3 fatty acids. The preparation and packaging of the nutritional supplement are done under reducing conditions such as in the presence of nitrogen, to avoid oxidation of the compound. The invention further describes the method of pre-treatment of tumor-bearing mammals with the nutritional supplement such that it precedes the initiation of, then is continued concomitant with, the therapy. The invention describes a way to protect normal organ function and reduce inflammation, as exemplified by the gastrointestinal tract, liver, and bone marrow upon chemotherapy treatment. The invention describes a formulation and method for use that enhanced general well-being and normal behavior of the treated mammals.

### CROSS-REFERENCE TO RELATED APPLICATIONS

This is a utility patent application that follows on Provisional Patent #60/278,138. It is related to PCT Patent #16666. Co-Inventors: Mary Pat Moyer, W Elaine Hardman, and Ivan L. Cameron.

### STATEMENT REGARDING FEDERALLY SPONSORED RESEARCH OR DEVELOPMENT

Parts of this research were sponsored by the National Institutes of Health of the U.S. Public Health and Human Services, thus providing government use of said invention according to law.

### REFERENCE TO A SEQUENCE LISTING, A TABLE, OR A COMPUTER PROGRAM LISTING COMPACT DISC APPENDIX

Not applicable

### BACKGROUND OF THE INVENTION

This invention relates to the preparation and use of nutritional supplements, in particular “accelerated action fatty acids” (AAFAs) prepared as concentrates, to minimize the side effects of chemical and radiation therapies and improve outcomes of said therapies in humans and other mammals.

A. Introduction

The public has becoming increasingly aware of the beneficial effects of dietary modifications to general health. This has included a general understanding that fats from different natural nutritional sources differ in their effects on health. As an example, many people have replaced complex, saturated animal fats in their diets by polyunsaturated vegetable fats for health reasons, particularly in an attempt to control serum cholesterol levels. Most recently, fish oils have been suggested as a dietary supplement for cholesterol and triglyceride control and antithrombotic benefits.

Fish oils are predominantly comprised of lipids, which are primarily long chain polyunsaturated fatty acids (PUFAs). These PUFAs can be classified into three major groups: .omega.3, .omega.6 and .omega.9. The classes are based on the location of the double bond closest to the methyl end of the fatty acid. If the closest double bond is between the third and fourth carbon atoms from the methyl group, the molecules are .omega.3 fatty acids. If the double bond is between the sixth and seventh carbon atoms, the molecules are classified as .omega.6 fatty acids. Man and other mammals cannot interconvert fatty acids from one family to another, but they can desaturate or elongate the fatty acid chains.

Most of the fatty acids consumed in normal nutrition have sixteen (C.sub.16) or eighteen carbon (C.sub.18) chains. The notation (C.sub.- - - :.sub.- - - .omega..sub.- - - ) indicates the number of carbon atoms in the chain, the number of double bonds, and the class of the fatty acid, respectively. The .omega.3 fatty acids are normally elongated and desaturated to form either the twenty carbon eicosapentaenoic (C20:5.omega.3) or the twenty-two carbon docosahexaenoic (C22:6.omega.3). The .omega.9 fatty acids are primarily elongated to form the twenty carbon eicosatrienoic (C20:3.omega. 9) while the most important twenty carbon .omega.6 fatty acid is arachidonic acid (C20:4.omega.6). Whether ingested or made in the body, the twenty or greater carbon PUFAs are the most important in terms of physiological functions.

PUFAs are recognized as important to general health and well-being, in particular in reference to the cardiovascular system. PUFAs are also recognized as important to the nutrition of critically ill patients, including cancer patients or those who may need to receive parenteral nutrition. These patients often have a poorly functional immune system and a high risk of infection. However, the formulations and methods for the use of PUFAs for nutrition and as an adjuvant to patient care, are highly variable. As an example, most currently available parenteral nutrition systems give much more of the essential FAs than is needed because they use soybean or safflower oil as the FA source. These oils contain primarily polyunsaturated .omega.6 fatty acids but have little or no twenty carbon length .omega.3 fatty acid content. Since parenteral nutrition diets supply between 10 and 50% of the calorie intake as oils, there is a large excess of .omega.6 FAs above the nutritional need of 2 to 4% of the total calorie intake as .omega.6 FAs.

In vitro studies indicate that addition of PUFAs from fish oil, including eicosapentanoic and docosahexanoic acids, to the culture media did increase the efficacy of the chemotherapeutic drug against different cancer cell types including: ZR-75-1 breast [Begin et al, 1986 J.Nat.Canc. Inst 77: 1053-1062; Begin et al., 1988, J.Nat.Canc. Inst 80: 188-194], transformed rat fibroblasts [Atkinson & Meckling-Gill, 1995, Cell. Pharmac. 2: 259-264], L1210 leukemic cells [deSalis & Meckling-Gill, 1995, Cell. Pharmac. 2: 69-74], A549 lung, PC-3 prostate [Begin et al, 1986 J.Nat.Canc. Inst 77: 1053-1062], THKE tumorigenic human kidney epithelial [Maehle et al. 1995,Brit. J. Cancer 71: 691-696], and MDA-MB 231 breast cancer cells [Hardman et al., 1997, Brit. J. Cancer 76: 347-354].

Although the use of PUFAs prior to beginning chemotherapy has been proposed as a way to sensitize cancers to the effects of a chemotherapeutic drug [Burns & Spector, 1987, Lipids 22: 178-184; Hardman et al., 1997, Brit. J. Cancer 76: 347-354; Shao et al., 1995, Lipids 30:1035-1045] the formulation and method of PUFAs use has not been clearly delineated so that those aware of the art have not reached a common consensus. Furthermore, the general mode for the preparation of PUFAs in oils usually includes the addition of an anti-oxidant during the manufacturing process. Also, in vivo studies on dietary PUFAs in concert with chemotherapy treatment in mammals have been limited with regard to the protective effects, thus not addressing the whole animal's physiological response to the treatment. Furthermore, the studies have not defined specific sources and delivery methods that can be obviously extrapolated to effective use.

In severely ill or stressed patients being treated by single or combinations of surgical therapy, chemotherapy or radiation therapy as a standard of care, there is a need to reduce the devastating side effects common with therapy. Accordingly, an object of the invention is to provide a method of minimizing the effects of treatment in at risk animals, particularly humans, by administering a diet that promotes resistance to, or recovery from, the side effects associated with treatment, disease-associated cachexia (wasting of the patient). Accordingly, an object of the invention is to provide a method of minimizing the effects of treatment in at risk animals, particularly humans, by administering a diet which enhances susceptibility of resistant cells to treatment and reduces the cachexia associated with disease.

Oils which are concentrated to provide a high percentage of .omega.3 fatty acids per unit volume are a preferred .omega.3 fatty acid source. A quantity of .omega.9 containing oils may also be present in the diet. Any diet within the scope of the invention might also include an amount of .omega.6 fatty acids to provide the essential fatty acids needed for good nutrition. Other nutrients, including vitamins and minerals, may be included in the diet for complete nutrition.

Patents in the Field

Patent searches were conducted using the Internet. Key words used in the various searches included combinations of: fatty acids, cancer, therapy, chemotherapy, fish oil, anticancer, normal cells, protection, etc. From those searches it was apparent that there are unique components of this invention as compared to prior art, public knowledge, and previously issued patents. Although these patents are in the field of use they do not focus on a concentrated product of the indicated formulation or on the multiple benefits in reducing the toxic side effects of chemotherapy while promoting normal, healthy cell and tissue growth.

Some examples of patents in the field of use are listed from the international patent search (PCT; www.wipo.org) or US Patent Search (www.uspto.gov) when “fatty acids” and “cancer” were used as key words to define records.

Patent Title Number

WO 02/16575 Method of Screening for Inhibitors of Human Fatty Acid-CoA Ligase 4

WO 02/13817 Remedies for Cisplatin-Tolerant Cancer

WO 02/03983 Fatty Acid Analogues for the Treatment of Cancer

WO 01/95859 Novel Heterocyclic Analogs of Diphenylethylene Compounds

WO 01/34202 Depletion of Cellular Coenzyme-A Levels as a Means to Selectively Kill Cancer Cells

WO 01/34145 Treating Cancer by Increasing Intracellular Malonyl COA Levels

WO 01/21172 Anti-Cancer Nitro- and Thia-Fatty Acids

WO 01/17524 Methods for treating Cell Proliferative Disorders Including Cancer

WO 00/53231 Fatty Acid Anticancer Conjugates and uses Thereof

WO 00/42832 New Compounds for the Treatment of Cancer

WO 00/33083 Diagnosis of Stage or Aggressiveness of Cancer

WO 99/48916 Hypoxia Inducible Human Genes, Proteins, and Uses Thereof

WO 98/41216 Composition for Prevention of Hepatic Steatosis

WO 98/09621 Fatty Acid Treatment

WO 98/08394 Method of Detecting Expression of and Isolating the Protein Encoded by the BRCA1 Gene

WO 98/06823 Cytokine-Free Culture of Dendritic Cells

WO 97/13415 Therapeutic Diet for Dogs with Lymphoma

WO 97/03663 Non-Oxidizable Fatty Acid analogues, Their uses as Therapeutic Active Medicaments, and Preparation Thereof

U.S. Patents

U.S. Pat. No. 6,326,355 Method for the Prevention and Treatment of Cachexia and Anorexia

U.S. Pat. No. 6,312,909 Composition and Methods for the Diagnosis Prevention and Treatment of Tumor Progression

U.S. Pat. No. 6,284,268 Pharmaceutical Compositions Containing an Omega-3 Fatty Acid Oil

U.S. Pat. No. 6,025,137 Compositions and Methods for the Diagnosis, Prevention and Treatment of Tumor Progression

U.S. Pat. No. 6,015,798 Method for Reducing the Damaging Effects of Radiation Therapy on Animal Skin and Mucosa

U.S. Pat. No. 5,968,896 Nutritional Supplement for Preoperative Feeding

U.S. Pat. No. 5,952,392 Long-chain Alcohols, Alkanes, Fatty Acids and Amides in the Treatment of Burns and Viral Inhibition

U.S. Pat. No. 5,547,927 Enteral Nutritional Product for Patients Undergoing Radiation Therapy and/or Chemotherapy

U.S. Pat. No. 5,514,656 Method of Providing Enteral Nutritional Support for Patients Undergoing Radiation Therapy and/or Chemotherapy

U.S. Pat. No. 5,336,485 Method for Protecting Animals Against Tacrine Induced Cytotoxic Injury Using Sterol Compounds

The formulation refers to the method of preparing the invention. This has key differences from prior art in that it specifically defines the need to prepare the oil as a concentrate according to the descriptions of the composition detailed below and to present it as a means to protect normal tissues and cells from damage of the chemical or radiation treatment. It is believed that these components of the formulation exploit the protection from enhanced lipid peroxidation and cell death, and increase the cell cycle time of normal cells, which are proposed as a mechanisms of action.

The method for use differs from prior art in that it defines a beneficial effect when used as an additive to traditional therapies at the equivalent concentration of 8 or more grams per day per patient, and that the use of the nutritional supplement is recommended to begin prior to the initiation of therapy with continued use during therapy, and maintenance concentrations thereafter. Thus, it is an integrated therapy.

### BRIEF SUMMARY OF THE INVENTION

This discovery optimizes methods for use of an omega.3 fatty acid formulation. The invention can be a physical mixture of the concentrated fish oil rich in .omega.3 fatty acids and freshly prepared as a food additive, or packaged as a gel capsule or liquid nutritional supplement under conditions that reduce oxygen, wherein handling and packaging by saturation with nitrogen gas is a preferred method.

Used with or without adjunct surgical therapies, chemotherapy in the form chemicals intended to kill or reduce the growth and spread (metastasis) of cancer cells is used in patients with cancer. For the purposes of this invention, “chemicals for chemotherapy” would not be limited to conventional anti-cancer agents but would include cytokines, chemokines, hormones, differentiation inducers, antibodies and any molecular-based therapeutics developed by a synthetic chemistry method, recombinant biotechnologies, or by isolation from natural products. Examples of standard chemotherapy agents would include members of the chemical families of camptothecin, doxorubicin, taxol, mitomycin, and cisplatin.

Used with or without adjunct surgical therapies, radiation therapy in the form of irradiation delivered by a targeted beam, radioactive pellets, or radioactive solutions intended to kill or reduce the growth and spread (metastasis) of cancer cells is used in patients with cancer. For the purposes of this invention, “radiation therapy” would not be limited to conventional beam radiation only but would include radioactive pellets or solutions.

Accordingly, an object of the invention is to provide a method of minimizing the toxic side effects of cancer therapy by supplementation of the diet of immunocompromised animals with tumors, particularly humans, with the fatty acids of fish oil.

Another object of the invention is to provide a dietary supplement which increases the efficacy of cancer treatment.

A further object of the invention is to provide a method of treating patients having a high risk of organ damage due to cancer treatment with a dietary supplement that provides fatty acids that improve general health of the organ.

Another object of the invention is the promotion of normal cell differentiation and function, with minimal induction of cell death by apoptosis or other mechanisms, as exemplified in studies of the gastrointestinal tract.

A still further object of the invention is to provide a lipid source and a dietary supplement useful in treating immunosuppressed patients, with diminished formation, differentiation or function of blood cells.

These and other objects and features of the invention will be apparent from the following description.

While the method and dietary supplement disclosed herein will not necessarily prevent or eliminate tumor growth, the reduction of side effects caused by the agents and methods used to treat the disease will promote survival of patients or animals with cancer. The use of an.omega.3 fatty acid-enriched oil without antioxidant provides not only the .omega.3 benefits of promoting survival from treatment effects but also the enhanced benefit of providing an important nutritional dietary component.

The specific method and dietary supplement set forth herein are purely illustrative and those skilled in the art may determine other modifications and variations of these procedures. Such other modifications and variations are included within the scope of the claims listed below.

### BRIEF DESCRIPTION OF THE SEVERAL VIEWS OF THE DRAWING

Not applicable

### DETAILED DESCRIPTION OF THE INVENTION

The following examples demonstrate descriptions of the invention and how it works.

### EXAMPLE 1

Toxicity of and Moderating Side Effects of CPT-11 Treatment by the Invention, Accelerated Action Fatty Acid (AAFA™), a Concentrated Omega 3 Fatty Acid Fish Oil Formulation

Summary

This example demonstrates that the invention showed protective and health-promoting effects in mammals treated with a chemotherapy drug, CPT-11. In humans, late diarrhea (>24 hours post CPT-11 treatment) associated with intestinal damage and decreased white counts, especially decreased neutrophil counts, are the main dose limiting side effects of treatment with CPT-11. The intestinal data indicate that consumption of the invention, AAFA, prior to and during CPT-11 treatment reduced intestinal damage, as reflected by less water in the feces and less CPT-11 induced cell death in the small intestine.

Increased serum glutamic oxalic transaminase (SGOT) levels, which indicate hepatotoxic damage, often follows CPT-11treatment. Enlargement of the liver as a result of chemotherapy may result from damage to the liver or to hypertrophy as a result of an increased demand to detoxify blood. In this example, the liver was a significantly larger fraction of the body weight of mice that were treated with CPT-11, and that did not consume AAFA, than in mice that were not treated with CPT-11. However, the liver fraction in mice that consumed AAFA prior to and during CPT-11 treatment was reduced to a level similar to the untreated mice.

Leukopenia or anemia occurs in 91% and 67% (respectively) of patients or other mammals treated with CPT-11. The data from this study indicate that consumption of AAFA by mice prior to and during CPT-11 treatment resulted in WBC and RBC counts that were similar to the WBC or RBC counts of untreated mice.

Numbers of micronuclei in the peripheral blood and apoptotic figures in the colon were used as assays of genetic damage in the blood-forming cells. Micronuclei are formed in blood cells following double strand breaks in DNA, apoptosis can result following unrepaired DNA strand breaks. These results indicate that there was less genetic damage in bone marrow and in the duodenum following CPT-11 treatment in the mice which consumed 2% or 3% AAFA. Cancer chemotherapeutic drugs typically cause more damage to faster proliferating cell populations than to slower proliferating populations. Omega-3 fatty acids have been shown to reduce proliferation in normal tissues. The reduced genetic damage in these normal tissues may be due to slower proliferation during the time of CPT-11 treatment.

Asthenia (tiredness or weakness) is reported by almost 90% of patients as a side effect of CPT-11treatment. The ‘scruffy’ look of the mice that did not consume AAFA and that were treated with CPT-11 is likely due to lack of grooming. This ‘scruffy’ look was decreased in mice which consumed 1% AAFA and completely eliminated in mice which consumed 2% AAFA probably indicating that they felt better and could continue normal grooming behavior.

There was a dose response towards normal in mice that consumed 1% or 2% dietary AAFA for fecal water, apoptotic figures in duodenum, liver weight, WBC and RBC parameters. The variance from the dose response for 3% AAFA in the diet may indicate that addition of more AAFA does not increase the effectiveness of the diet supplement or that the effective dose of AAFA was exceeded. Two percent AAFA in the diet of the mice is equal to 18 calories AAFA/430 calories diet or 4.1% of the calories in the diet from AAFA. In human terms, consumption of 9 grams per day in an 1800 calorie diet would be 81/1800 or 4.5% of the calories from AAFA, 8 grams per day would equate to 72/1800 or 4.0% of the calories from AAFA. Thus consumption of 8 to 9 grams per day by humans would equate to the 2% AAFA diet fed the mice in this study.

Aims of Study

The major aims of the study were (1) To determine if toxicity was associated with consumption of INCELL AAFA at levels of 1%, 2% or 3% of the diet in mice; and (2) To determine if 1%, 2% or 3% INCELL AAFA incorporated in the diet altered the side effects of CPT-11 treatment.

Experimental Design

40 male Swiss mice, 6 weeks old, were obtained and earmarked for unique identification. Mice were housed 5/cage in Laboratory Animal Resources facilities. After 24 hours acclimation, mice were weighed and the food was changed to one of four defined diets. The AIN-76 based, 10% total fat diets contained: 0% AAFA+10% corn oil, 1% AAFA+9% corn oil, 2% AAFA+8% corn oil or 3% AAFA+7% corn oil. Two cages (10 mice) were designated to consume each diet. All mice were weighed three times weekly throughout the experiment.

Mice consumed the diet for 2 weeks then treatment with CPT-11 commenced for half of the mice (1 cage of 5 mice) on each diet. CPT-11 was administered at 60 mg/kg body weight, IV in a lateral tail vein, once each four days for 2 weeks. Control mice received an equivalent volume of the CPT-11 vehicle.

Mice were killed 24 hours after the last CPT-11 treatment. At necropsy, mice were weighed, anesthesized and blood was drawn via cardiac puncture. Peripheral blood was placed in an EDTA-containing tube and sent to Laboratory Animal Resources for complete blood counts. The liver, spleen, testes and kidneys were removed, weighed, then part of each organ was fixed in Omnifix® and a part was quick frozen in liquid nitrogen. The brain was removed and frozen in liquid nitrogen. The colon was resected, split open longitudinally and placed serosal side down on a card. Feces were removed from the colon, placed in a tared pan and weighed, then placed in a 100° C. vacuum oven for drying to constant weight. The colon and a section of duodenum were fixed in Omnifix®. The fixed colon and duodenum were oriented in cassettes then processed to paraffin blocks. Paraffin sections were cut 4 μm thick, histological slides of the colon and duodenum were prepared and stained with H & E or periodic acid-Schiff stain.

After completion of blood counts, a thin smear of peripheral blood was made on a microscope slide, the smear was air-dried and was then stained for 5 minutes with 0.01 mg/ml acridine orange in phosphate buffered saline (pH 7.0). Micronuclei and polychomataphilic erythrocytes (PCE) were identified on the acridine orange stained smears of peripheral blood. Using fluorescent microscopy and a 100X oil objective, the mature erythrocytes, lacking either DNA or RNA are green, the RNA containing PCE are reddish and the DNA containing micronuclei are bright yellow. Fields containing a single layer of erythrocytes were identified. At least 8 standard fields (300, 400 or 500 cells) were identified on each slide and the number or micronuclei or PCEs were counted and recorded.

Statistical Analyses

All data was analyzed by two-way or one-way analysis of variance followed by Student-Newman-Keuls multiple range test as appropriate. Data is presented graphically, the Y-axis of each graph is labeled to identify each set of data. On each graph, groups which do not share a superscript are significantly different (p<0.05).

Drug Preparation

CPT-11 was obtained as irinotecan hydrochloride (Pharmacia & Upjohn, Kalamazoo, Mich.). It was prepared, according to manufacturer's directions, to duplicate the clinical formulation (Camptosar™). The prepared CPT-11 contained: 20 mg/ml irinotecan hydrochloride, 45 mg/ml sorbitol (Sigma, St. Louis, Mo.) and 0.9 mg/ml lactic acid (Sigma, St. Louis, Mo.) and was pH adjusted to 3-3.8. The solution was warmed in a 100° C. water bath to dissolve the CPT-11. A dose of 60 mg CPT- 11/kg body weight (about 0.08 ml/ 28 g mouse) was injected into the lateral tail vein of each treated mouse, once each 4 days for 2 weeks.

Diet Preparation

Dry ingredients were measured and mixed well, then oil (as a mixture of AAFA and corn oil) was added at 10% of the weight of the dry ingredients. Water was added as needed to hold ingredients together. Food was stored at −20° C. until fed to the mice. Mice were fed fresh food every afternoon, seven days/week.

| Diet composition | Dry mix (g/95 g) | Final composition (g/100 g) |
|---|---|---|
| Total oil | | 10 |
| Sugar | 50 | 47.36842 |
| Casein | 20 | 18.94737 |
| Cornstarch | 15 | 14.21053 |
| AIN-76 vitamin mix | 1 | 0.947368 |
| AIN-76 mineral mix | 3.5 | 3.315789 |
| Choline bitartrate | 0.2 | 0.189474 |
| DL-methionine | 0.3 | 0.284211 |
| Cellulose | 5 | 4.736842 |
| Total | 95 | 100 g |

Results

Body weight

There were no significant differences in the mean body weight of the groups of mice at the beginning of CPT-11 treatment. During the time of CPT-11 treatment, mice treated with CPT-11 gained less weight than the mice which did not receive CPT-11 however the results of ANOVA revealed that the weight gained during the time of CPT-11 treatment was not significantly different between mice which did and which did not receive CPT-11. There were no significant differences in weight gained due to the diets of the mice.

Fecal Water

The initial wet weight and final constant weight of the dried feces was used to calculate the % of water in the feces of each mouse as an assay of intestinal function. As shown in graph 1, the mean fecal water content of mice that were treated with CPT-11 and that did not consume AAFA was higher than the fecal water content of mice not treated with CPT-11. Due to the small numbers, the fecal water results were not significantly different by ANOVA, however, the mean fecal water content of mice that consumed 1% or 2% AAFA and that were treated with CPT-11 was less than the fecal water content of mice which did not consume AAFA and was at the level of the untreated (normal) mice. These results tend to indicate that large bowel function was preserved in mice which consumed AAFA. Thus, the treatment helped return the values closer to control (no CPT-11 treatment) group values.

Apoptotic Figures in the Duodenum

The number of apoptotic cells found in 10 complete, midaxially sectioned duodenum crypts was counted as an indication of intestinal damage. The results of two way ANOVA (graph 2) revealed that CPT-11 significantly increased the number of apoptotic cells in the duodenum, however, consumption of 3% AAFA significantly decreased the number of apoptotic cells. Thus, the treatment helped return the values closer to control (no CPT-11 treatment) group values.

Liver Weight

The weight of the liver was expressed as a percent of body weight. As illustrated in graph 3, ANOVA of the liver weights revealed that the livers of the mice that were treated with CPT-11 and that did not consume AAFA were significantly larger than the livers of mice not treated with CPT-11. However, the livers of mice that consumed AAFA and that were treated with CPT-11 were not significantly larger than livers of the untreated mice. Thus, the treatment returned the values to control (no CPT-11 treatment) group values.

Blood Counts

Graphs 4, 5 and 6 illustrate the results of ANOVA of the white blood cell and red blood cell counts and the hematocrits of the mice. Treatment with CPT-11 significantly decreased all three measures. However, consumption of AAFA prior to and during CPT-11 treatment resulted in higher WBC, RBC and hematocrit than in mice which did not consume AAFA. The WBC and RBC counts of mice that consumed 2% AAFA and that were treated with CPT-11 were not significantly different from the WBC or RBC counts of mice which did not receive CPT-11. Thus, the treatment returned the numbers to control (no CPT-11 treatment) group values.

Micronuclei and Polychromataphilic Erythrocytes

Graphs 7 and 8 illustrate the results of counts of micronuclei (MN) and polychromataphilic erythrocytes (PCE) in the peripheral blood. In mice that did not consume AAFA, both MN and PCE were significantly higher following treatment with CPT-11 than in mice that did not receive CPT-11 treatment. However, consumption of 2% or 3% AAFA prior to and during CPT-11 treatment resulted in numbers of MN and PCE which were not significantly different than in the mice which did not receive CPT-11. Thus, the treatment helped return the values closer to control (no CPT-11 treatment) group values.

Coat Quality

The group of mice that consumed the ‘normal’ diet containing 10% corn oil and 0% AAFA that were not treated with CPT-11 (normal control group) was compared to the groups of mice treated with CPT-11 that consumed each diet. It was observed that the coats of the mice that did not consume AAFA and that were treated with CPT-11 look ‘scruffy’, i.e., the fur is not smooth and hair strands were sticking together so that underlying skin showed through, especially between the scapulae. The coats of mice treated with CPT-11 that consumed 1% AAFA look somewhat smoother, the coats of mice that consumed 2% or 3% AAFA did not look different from the mice that did not receive CPT-11. Thus, the treatment helped return the animal grooming behavior closer to the normal untreated control group without CPT-11. This generally reflects that the animals were “feeling better”.

### EXAMPLE 2

Summary

The invention can be prepared as a dietary supplement either as a food additive or a separately manufactured supplement. It can be used after tumors are growing in the host. Its use should precede the beginning of therapy by at least one week, and preferably 2 weeks, then be continued for the duration of the treatment. When used as a food additive it will need to be replaced daily to avoid decomposition. When the invention was used in this manner, and as detailed in the Methods and Materials below, tumor growth curves revealed that supplementation of the diet with either the 3% or 6% w/w fish oil fed prior to and during treatment with CPT-11 enhanced regression of the MCF7 human breast cancer xenografts in nude mice. Evaluation of side effects of CPT-11 treatment showed that CPT-11 treatment caused the same body weight loss whether the mice were fed the corn oil or the fish oil diets however, histopathological damage to the small and large intestine was reduced when the CPT-11 treated mice consumed dietary fish oil. Thus, consumption of low levels of dietary fish oil increased the efficacy of CPT-11 against the tumor and decreased the histopathological damage to the intestines caused by CPT-11 treatment. In this preclinical study, supplementation of the diet with low levels of fish oil proved to be an effective adjunct to chemotherapy with CPT-11 and may also be an effective adjunct to cancer chemotherapy with CPT-11 in humans.

Methods and Materials

Tumor Cells

MCF7 human breast cancer cells (American Type Culture Collection, Rockville, Md.) were cultured for injection in nude mice. The culture medium was M3:10™ (INCELL Corporation, LLC).

Mice

Twenty female nude mice (nu/nu, Harlan Sprague Dawley, Madison, Wis.) 6 weeks old were used in this study. Each mouse was numbered for identification. The mice were allowed to acclimate for one week before beginning the experiment. Mice were housed under aseptic conditions (positive air pressure in a designated nude mouse room, cages, bedding, and water cages had microisolator tops) in a temperature (24° C.) and light controlled (12 h light per day) room. All mouse handling was carried out under a laminar flow hood. All animal use and handling was approved by the UTHSCSA Institutional Animal Care and Use Committee before commencing the experiment. The animal care facilities are accredited by the American Association for the Accreditation of Laboratory Animal Care.

Estrogen Supplementation

Female nude mice produce inadequate estrogen to support the growth of MCF7 cells. Therefore, the mice were given injections of beta. estradiol (Sigma, St. Louis, Mo.) dissolved in pure sesame oil (0.1 mg/0.05 ml sesame oil per mouse, s.c. over rump) beginning one day before the injections of MCF7 cells and at weekly intervals thereafter until the end of the experiment.

Preparation of Cells

Cultured MCF7 cells were harvested, rinsed then suspended in serum-free M3D base culture medium (INCELL Corporation, LLC). Cells in suspension were counted using a hemocytometer and the cell count was adjusted to 10⁸/ml. The suspension was kept well mixed during the time of injection. MCF7 cells (5×10⁶ cells in 0.05 ml of serum free media) were injected sc on the upper back of each mouse.

Experimental Design

Mice were fed Harlan Teklad LM-485 Mouse Chow diet while the tumors were allowed to grow to about 5 mm diameter. This allowed the tumors to become established as growing tumors in the host mice before feeding of the experimental diets. The tumor bearing mice were then divided into four groups. One group of five mice remained on the chow diet and did not receive CPT-11 (untreated, normal control). Three groups were placed on diets, based on the AIN-76A diet, modified to contain 7% total fat.

The 7% total fat for each of the three experimental diets was divided as follows: 1) 7% corn oil, 0% fish oil (control diet with CPT-11), 2) 4% corn oil, 3% antioxidant-free fish oil (3% fish oil with CPT-11) or 3) 1% corn oil, 6% antioxidant-free fish oil (6% fish oil with CPT-11). The compositions of the experimental diets are shown in Table 1. Fish oil was purchased and used without added antioxidant specifically to exploit the potential to enhance lipid peroxidation, as in our previous study (Hardman et al., 1997). The corn oil and/or fish oil diets were prepared weekly, daily portions for each cage were packaged individually and the packages were stored in sealed containers at −20° C. to suppress spontaneous lipid peroxidation. The corn oil and the fish oil diets were replaced daily. The mice were maintained on these diets for ten days before beginning treatment with CPT-11.

Drug Preparation

CPT-11 was obtained as irinotecan hydrochloride (Pharmacia & Upjohn, Kalamazoo, Mich.). It was prepared, according to manufacturer's directions, to duplicate the clinical formulation (Camptosar™). The prepared CPT-11 contained: 20 mg/ml irinotecan hydrochloride, 45 mg/ml sorbitol (Sigma, St. Louis, Mo.) and 0.9 mg/ml lactic acid (Sigma, St. Louis, Mo.) and was pH adjusted to 3-3.8. The solution was warmed in a 100° C. water bath to dissolve the CPT-11. A dose of 60 mg CPT-11/kg body weight (about 0.08 ml/ 28 g mouse) was injected into the lateral tail vein of each treated mouse, once each 4 days for 6 weeks.

Tumor and Body Weight Measurements

Lengths and widths of tumors and body weights were measured three times weekly. Measurements were entered directly into an Excel spreadsheet. Tumor sizes were calculated using the formula for the volume of a prolate spheriod: V=4/3*3.14*L/2*W/2*D/2. The width measurement was used as the depth of the tumor. This shape was a good approximation of the shape of the tumors.

The experiment was terminated 28 days after the initiation of CPT-11treatment. The mice were anesthetized using a ketamine/S. A. rompun mixture (0.2 cc/25 g weight, IM; prepared by our Laboratory Animal Resources veterinarian), then killed by cervical dislocation.

Necropsy and Tissue Processing

Tumors, liver, small and large intestines were removed at necropsy. Tumors were fixed in Omnifix™ (Melville, N.Y.) for later analysis. Omnifix™ is an alcohol-based, proprietary formula fixative which does not crosslink antigen epitopes as does formalin. A portion of the liver was flash frozen in liquid nitrogen for later analyses. A one cm segment of each small intestine and a one cm segment of each large intestine were consistently removed from regions 2 cm from the stomach/duodenum junction or 4 cm from the anus, respectively. These tissue segments were placed on a small piece of cardstock, split longitudinally, spread and affixed mucosal side up to the card. Card and tissue were then placed in Omnifix™ for fixation. After fixation, tissues from individual mice were placed in a tissue cassette and processed for embedding in paraffin blocks. Small and large intestine segments were oriented on-edge in the paraffin blocks so that complete longitudinally sectioned crypts would be seen on microscope slides. Four μm thick sections were cut and placed on slides. One set of slides was stained using hematoxylin and eosin (H&E), a second set of slides was stained by the periodic acid-Schiff (PAS) reaction and counterstained with hematoxylin to identify mucin in goblet cells. Slides were coded and evaluated by an observer blinded to the group of origin of each slide.

Products of Lipid Peroxidation

At a later date, frozen livers were thawed and homogenized individually at 4° C. using a Polytron homogenizer. The total protein content of an aliquot of the whole specimen homogenate was analyzed by the method of Bradford (Bradford, 1976) using the Bio-Rad protein assay (micro-method). The thiobarbituric acid reactive substances (TBARS) assay was used to estimate lipid peroxidation on the remainder of the homogenate. Malondialdehyde and other products of lipid peroxidation can be estimated spectrophotometrically at 535 nm after reaction with thiobarbituric acid to obtain an index for lipid peroxidation (Esterbauer et al., 1991). We realize that TBARS does not measure all products of lipid peroxidation and that there may be minor interference by other substances (sugars, amino acids, etc.), however, this simple inexpensive test does provide a good estimate of changes in overall lipid peroxidation of tissues. The absorbance values obtained were compared against a standard curve of known concentrations of malondialdehyde and normalized by protein content of the specimen. The results were reported as nmol of TBARS per mg of protein.

Histological Analyses of Duodenum and Colon

Only complete midaxially sectioned crypts in duodenum and colon on H&E stained slides were selected for analyses of crypt height, and number and location of mitotic and apoptotic cells. Complete crypts were defined as those with: 1) the crypt base at the muscularis mucosa, 2) an open lumen from mouth to base and 3) a single column of epithelial cells up each side of the crypt.

Crypt height was defined as the number of cells in a single column from the center of the base to the mouth of the crypt in complete midaxially sectioned crypts. Mitotic and apoptotic figures in the duodenum were identified on hematoxylin and eosin stained slides. The position of each mitotic or apoptotic figure in number of cells from the center of the crypt base were recorded. Apoptotic events were identified by the morphological parameters of nuclear marginalization of the chromatin, condensation of the cytoplasm, cell shrinking, membrane blebbing and finally fragmentation of the cell into apoptotic bodies (Potten, 1992; Barnes et al., 1997).

PAS stained slides were used to determine the distribution of mucin containing goblet cells. Complete, midaxially sectioned crypts in sections of duodenum and colon were identified then the location of each PAS stained cell (PAS⁺) in number of cells from the center of the crypt base to the crypt mouth was recorded.

The thickness of the muscularis mucosa was measured on the H&E stained sections using a calibrated ocular micrometer.

Statistical Analyses

Mean body weights during the experiment were analyzed by linear regression analyses using PRISM^{□}. Body weight data were divided into two linear regression analyses: 1) the time before the initiation of CPT-11 and 2) the time after initiation of CPT-11 to determine the effect of the diet and of the CPT-11 on the rate of change of the mean body weights of each group of mice. Significant differences between the slopes of the linear regression analyses either before or after CPT-11 treatment were determined by analysis of variance (ANOVA) followed by a Student-Newman-Keuls multiple range test (SNK) using PRISM™ software.

The mean tumor volume for each group was normalized to zero at the beginning of the CPT-11 treatment. Mean tumor growth curves were generated for each group and linear regression analysis was used to assess the tumor growth rate of each group. A significant positive slope indicated tumor growth, a significant negative slope indicated tumor regression and a non-significant slope indicated no growth. Analyses for differences between slopes of the regressions of the mean tumor volumes of each group were performed by PRISM™ (Graphpad Software, San Diego, Calif. ) using the general linear model to generate an ANOVA. An SNK multiple range test was used to determine which slopes were significantly different against the null hypothesis that there was no difference between the slopes. A probability value (p)<0.05 was used to indicate that the tumor growth rates represented by the slopes were significantly different.

ANOVA followed by SNK was used to determine differences between groups in TBARS in the livers.

Kolmorogov-Smimov (K-S) tests for normality showed that the distributions of the heights of mitotic and apoptotic figures in duodenum crypts differed significantly from a normal distribution. However, the distribution of the square root transformed heights of mitotic and apoptotic figures did not differ significantly from a normal distribution. Thus, a parametric ANOVA followed by an SNK was used to test for differences between the means of the square root transformed heights of mitotic figures or of apoptotic figures.

The K-S test showed that the crypt heights and the locations of PAS positive cells along the length of the crypt were not significantly different from a normal distribution, thus transformation was not needed prior to ANOVA of crypt heights or of means of locations of PAS positive cells. A p<0.05 was used to indicate that the means of two groups were significantly different.

Results of These Observations are Summarized as Follows

Results—Mouse Body Weights

The slopes of the linear regression analyses (Table 2) of the mean body weights (rate of change in body weight) per group for days 1 to 14 (prior to any drug treatment) were determined to provide information about the effect of diet on the mouse body weight. The effect of treatment with CPT-11 on body weight was determined from the mean body weights for days 14 to 42 of the study. Results are shown in Table 2.

Results—Mean Tumor Size

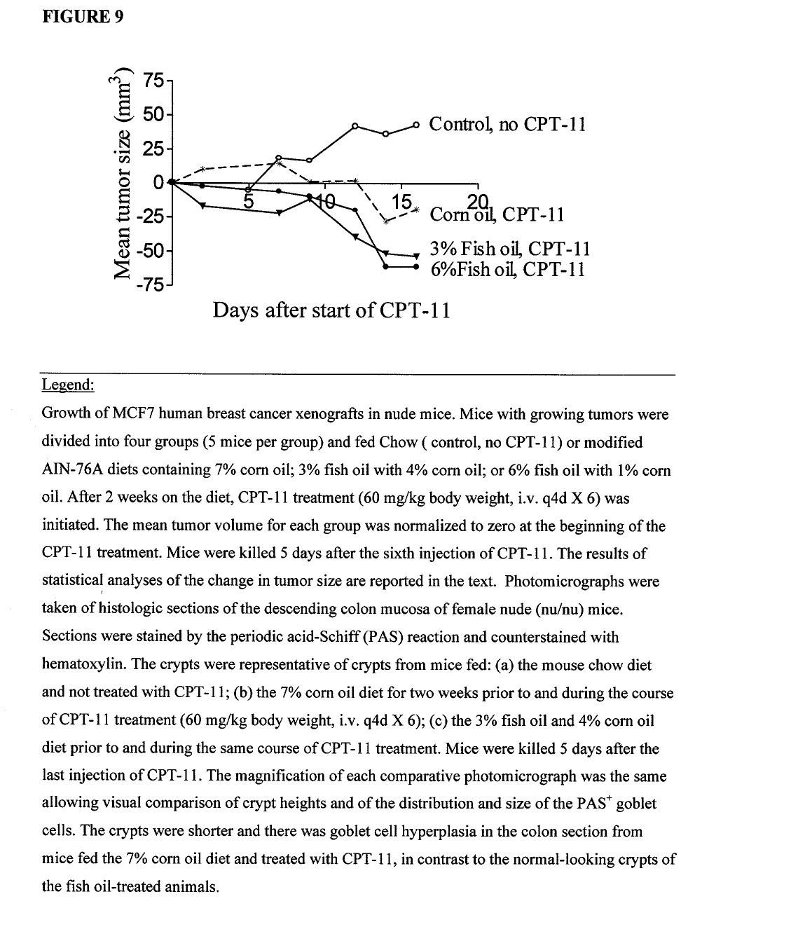

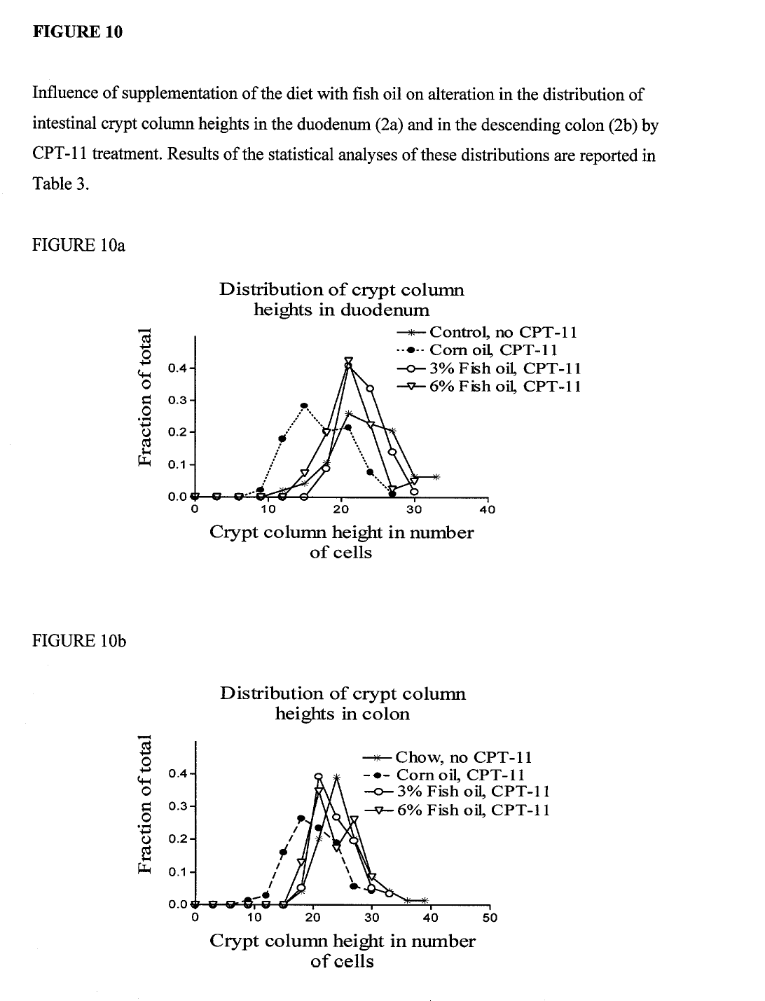

The mean tumor sizes over the time of treatment are presented in FIG. 9. The slope of the linear regression analyses (tumor growth rate in mm³/day) for the control group which did not receive CPT-11 was significantly positive (indicating continued tumor growth, slope=3.2±0.7) and was significantly different from the tumor growth rates of the three groups of mice which received CPT-11. The tumor growth rate of the group of mice treated with CPT-11 and fed 7% corn oil (slope=−1.8±0.8) was not significantly different from a slope of zero (indicating that growth of the tumor was halted by the CPT-11 treatment in this group of mice) and the tumor growth rate in the 7% corn oil fed group was significantly different from that of the other three groups. The mean tumor growth rates of the groups treated with CPT-11 and fed 3% fish oil (slope=−3.1±0.6) or 6% fish oil (slope=−3.9±1.0) were: 1) significantly negative, indicating significant regression of the tumor, 2) not significantly different from each other, 3) significantly different from the mean tumor growth rate of the mice which did not receive CPT-11 and 4) significantly different from the mean tumor growth rate of the mice which were treated with CPT-11 and fed 7% corn oil.

Results—Thiobarbituric Acid Reactive Substances

ANOVA revealed that when mice were killed five days after treatment with CPT-11, there was not a significant difference in the nmol TBARS/mg protein in the livers of the mice due to treatment with CPT-11 or to the diet of the mice (data not shown). Tumors of the CPT-11 treated and fish oil fed groups had regressed to the extent that there was not enough tumor tissue for TBARS analyses.

Histomorphometric Analyses of Duodenum and Colon

Results—Crypt Column Heights

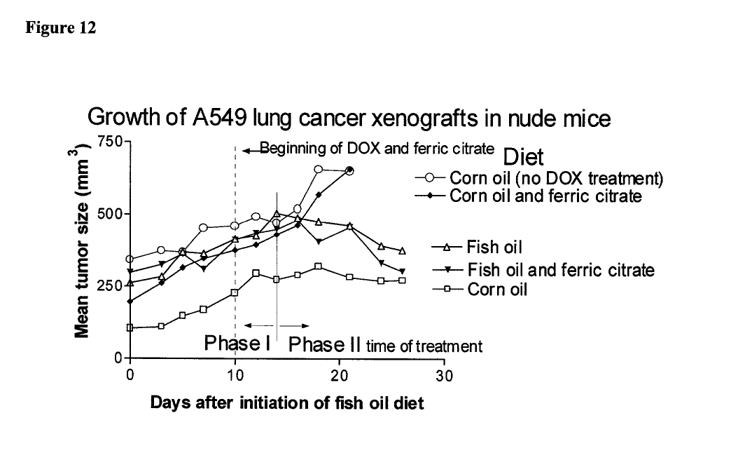

Graphs of the distributions of the crypt heights in the duodenum (FIG. 12a) and colon (FIG. 12b) illustrate that the mean of the distribution of crypt heights of the group treated with CPT-11 and fed 7% corn oil was less than the means of the other three groups. The distributions of crypt heights of the groups treated with CPT-11 and fed either 3% or 6% fish oil were similar to that of the group which did not receive CPT-11 treatment.

ANOVA followed by SNK of the crypt column height data (Table 3) revealed that, when compared to the control group which did not receive CPT-11:

1. the mean crypt column heights in the duodenum and colon were significantly less in mice fed 7% corn oil and treated with CPT-11

2. the mean crypt column heights in the duodenum and colon were not significantly less in mice fed 3% fish oil and treated with CPT-11

3. the mean crypt column height in the colon was not significantly less in mice fed 6% fish oil and treated with CPT-11.

ANOVA also revealed that the crypt column heights in both the duodenum and colon of mice fed either 3% or 6% fish oil and treated with CPT-11 were significantly greater than in mice fed 7% corn oil and treated with CPT-11.

Results—Mitotic Figures

ANOVA of the numbers of mitotic figures per midaxial crypt section in the duodenum (Table 4) revealed that there was not a significant difference between groups. However, ANOVA followed by SNK of the square root transformed heights of the mitotic figures revealed that there was a significant difference between groups in the distribution of mitotic figures in the duodenum. Specifically, the mean of the distribution of the heights of mitotic figures in the duodenum of the group treated with CPT-11 and fed corn oil was significantly less than the mean of the distribution in the mice which did not receive CPT-11 or in the mice treated with CPT-11 and fed 3% fish oil. The distribution of mitotic figures in the duodenum of the groups which were treated with CPT-11 and fed either 3% or 6% fish oil was similar to the group which did not receive CPT-11. There were too few mitotic figures in the histologic sections of colon for meaningful statistical analysis.

Results—Apoptotic Figures

ANOVA followed by SNK revealed that the number of apoptotic figures per midaxial crypt section in the duodenum of the group treated with CPT-11 and fed corn oil was significantly higher than in the control group which did not receive CPT-11 (Table 4). The mean number of apoptotic figures in the groups treated with CPT-11 and fed 3% or 6% fish oil was intermediate and not significantly different from either the control group which did not receive CPT-11 or the group treated with CPT-11 and fed corn oil.

The mean height of the distribution of apoptotic figures in the duodenum of the group treated with CPT-11 and fed corn oil was significantly less than the mean of that distribution in the mice which did not receive CPT-11 or in the mice treated with CPT-11 and fed 3% fish oil, as summarized in Table 4. There were too few apoptotic figures in the histologic sections of colon for meaningful statistical analysis.

Results—PAS⁺ Goblet Cells

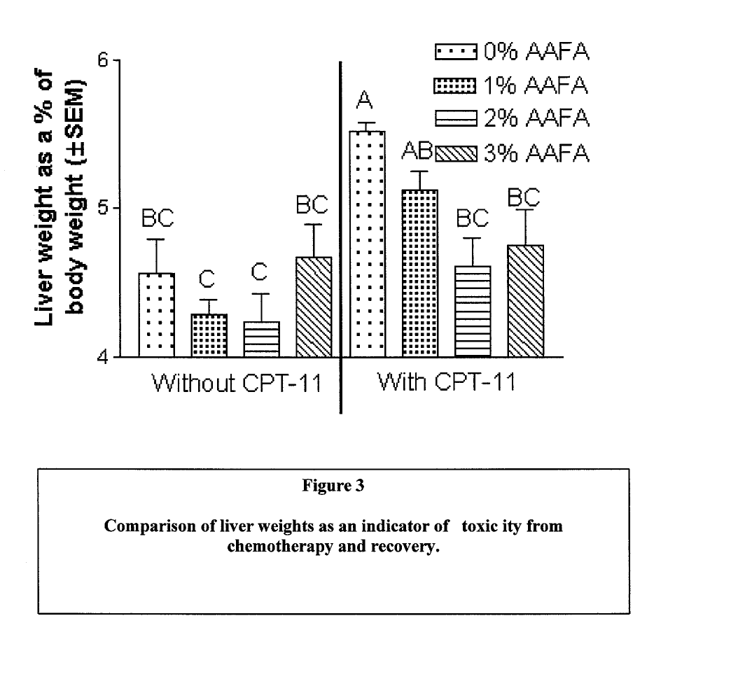

ANOVA followed by SNK revealed that the mean number of PAS⁺ goblet cells per colon crypt column in the group of mice treated with CPT-11 and fed the 7% corn oil diet, (Table 5) was significantly higher than in the group of mice which did not receive CPT-11. However, in the groups of mice fed the 3% or 6% fish oil diet and treated with CPT-11, the mean number of PAS⁺ goblet cells per colon crypt was not significantly different from that of the control group of mice which did not receive CPT-11. Examples of these findings are depicted in the photomicrographs in FIG. 3. The mean height of PAS⁺ goblet cells in the colon crypts was significantly lower in all groups of mice treated with CPT-11 than in the group of mice not treated with CPT-11.

Statistical analyses by ANOVA followed by SNK revealed that in groups of mice treated with CPT-11, the mean number of goblet cells per crypt column in the duodenum (Table 5) was significantly less than in the mice which did not receive CPT-11. However, the mean height of PAS⁺ goblet cells in the duodenum crypts of mice fed 7% corn oil or 3% fish oil diets and treated with CPT-11 was not significantly different from that of the control group of mice which did not receive CPT-11.

Results—Thickness of Muscularis Mucosa

Statistical analyses by ANOVA followed by SNK revealed that the thickness of the muscularis mucosa layer in the colons of mice treated with CPT-11 and fed the diet containing 6% fish oil (10.8±0.2 μm) was not significantly different from that of the mice not treated with CPT-11 (11.1±0.3 μm). However, the thickness of the muscularis mucosa layer in the colons of mice fed 7% corn oil and treated with CPT-11 (6.6±0.2 μm) was significantly less than in the mice not treated with CPT-11 or the mice fed 6% fish oil and treated with CPT-11. The thickness of the muscularis mucosa layer in the colons of mice fed 3% fish oil and treated with CPT-11 (7.7±0.2 μm) was intermediate in thickness but was not significantly differently from the mean value of the other groups. The muscularis mucosa layer in the duodenum of all groups was too thin for reliable measurements.

Results—Liver Membrane Fatty Acids

Consumption of 2% AAFA™ significantly (p<0.01) increased the fraction of EPA and DHA in liver cellular membranes. The EPA and DHA fractions (mean±SE) were 0.08±0.06% and 0.57±0.27 respectively in mice fed 0% AAFA/10% CO diet but the EPA and DHA fractions were 4.5±1.5% and 3.92±0.30%, respectively, in mice fed the 2% AAFA/8% CO diet. The AA fractions (range 8-10%) were not significantly different due to the diet of the mice.

TABLE 1
Composition of the experimental diets by weight percent (g/100 g of food)

| Ingredient | |
|---|---|
| ^{a}Total oil | 7.0 |
| Sugar | 48 |
| Casein | 20 |
| Cornstarch | 15 |
| AIN-76 vitamin mix^{a} | 1.0 |
| AIN-76 mineral mix^{a} | 3.5 |
| Choline bitartrate | 0.2 |
| DL-methionine | 0.3 |
| Cellulose | 5 |
| Total | 100.0 |
| Composition of the diets by % | |
| calories^{b} | |
| Protein | 20.2 |
| Carbohydrate^{c} | 63.9 |
| Fat | 15.9 |
| ^{b}Energy content of each diet, kcal/g | 3.95 |
| Footnotes to Table 1: | |
| ^{a}Total fat was 7%. Diet types included 7% corn oil (control diet with CPT-11) or 4% corn oil, 3% antioxidant-free menhaden fish oil (3% fish oil diet) or 1% corn oil, 6% antioxidant-free menhaden fish oil (6% fish oil diet). | |
| ^{b}Caloric content is calculated at 4 kcal/g for protein and carbohydrate and 9 kcal/g for fat. | |
| ^{c}The % of calories from carbohydrate include the calories from sucrose, cornstarch and sucrose in the vitamin and mineral mix. Diet components and chemicals - Purified high nitrogen casein, pure corn starch, Alphacel (non-nutritive bulk cellulose) AIN-76 vitamin mixture, AIN-76 mineral mixture and choline bitartrate (99% pure) were obtained from ICN Nutritional Biochemicals, | |
| # Cleveland, Ohio. Imperial brand (Sugarland, TX.) extra fine pure cane sugar and 100% pure corn oil (Wesson) were purchased locally. D.L. methionine (cell culture, M. W. 149.2), antioxidant free menhaden fish oil and ferric citrate were purchased from Sigma, St. Louis, Missouri. | |

TABLE 2
Slopes of the linear regression analyses of mouse body weights
(g change per day) following dietary modification and CPT-11 treatment^{a}.

| | Days 1-14 after dietary | Days 14-33 after dietary |
|---|---|---|
| Diet | modification (mean ± SD) | modification (mean ± SD) |
| Control, no | −0.049 ± 0.028 | no CPT-11, 0.22 ± 0.024 |
| CPT-11 | | |
| 7% Corn oil | −0.010 ± 0.042 | with CPT-11, −0.047 ± 0.020 |
| 3% Fish oil | 0.064 ± 0.048 | with CPT-11, −0.116 ± 0.020 |
| 6% Fish oil | 0.038 ± 0.036 | with CPT-11, −0.067 ± 0.016 |
| ^{a}During days 1-14, no slope was significantly different from a slope of zero (no change in body weight per day). During the time of CPT-11 treatment (60 mg/kg body weight CPT-11 each 4 days) all CPT-11 treated groups showed significant weight loss. ANOVA followed by SNK multiple range test of the slopes showed that there was no significant difference in the slope (weight loss) | | |
| # due to the diet of the CPT-11 treated mice. | | |

EXAMPLE 2 TABLE 3
Influence of dietary fish oil on alteration of intestinal crypt column height^{a}
by CPT-11 treatment.

| Diet | | | Duodenum | | Colon |
|---|---|---|---|---|---|
| 5 mice/group | CPT-11 | n^{b} | mean ± SD | n^{b} | mean ± SD |
| Chow | − | 92 | 23.5 ± 5.0^{d} | 70 | 25.0 ± 4.0^{c} |
| 7% Corn oil | + | 90 | 17.1 ± 4.0 | 66 | 20.1 ± 4.5 |
| 3% Fish oil | + | 74 | 22.6 ± 2.6^{d} | 56 | 23.7 ± 3.3^{c} |
| 6% Fish oil | + | 64 | 21.0 ± 3.2^{c} | 23 | 23.3 ± 3.8^{c} |

^{a}Crypt column height is expressed in number of cells from the base to the mouth of the crypt
^{b}n = total number of crypt columns counted per group
^{cd}ANOVA followed by SNK multiple range test showed that means which share a superscript in a column are not significantly different.

EXAMPLE 2 TABLE 4
Influence of CPT-11 treatment dietary fish oil on the number and the
location of mitotic and apoptotic figures in duodenum crypts.

| | | | | Trans. | | | Trans. |
|---|---|---|---|---|---|---|---|
| | | N | | height | N | | height |
| Diet | | mitotic | Mitotic | mitotic | apoptotic | Apoptotic | apoptotic |
| 5 mice/group | CPT-11 | figures^{a} | figures/crypt^{b} | figures^{b} | figures^{a} | figures/crypt^{b} | figures^{c} |
| Chow | − | 101 | 2.2 ± 1.5^{d} | 3.1 ± 0.9^{d} | 52 | 0.46 ± 0.19^{d} | 2.79 ± 0.95^{d} |
| Corn oil, | + | 93 | 2.1 ± 1.5^{d} | 2.5 ± 0.7^{e} | 58 | 1.14 ± 0.49^{e} | 2.03 ± 0.76^{e} |
| 3% Fish oil | + | 73 | 2.6 ± 1.7^{d} | 2.9 ± 0.8^{d} | 88 | 0.74 ± 0.22^{de} | 2.76 ± 0.81^{d} |
| 6% Fish oil | + | 40 | 2.0 ± 0.9^{d} | 2.6 ± 0.6^{e} | 80 | 0.90 ± 0.28^{de} | 2.30 ± 0.83^{e} |

^{a}Total number of mitotic or apoptotic figures counted per group
^{b}Mean number of mitotic or apoptotic figures per midaxial crypt section (mean ± SD).
^{c}Mean of the square root transformed heights of mitotic or apoptotic figures (mean ± SD).
^{de}Means that share the same superscript in a column are not significantly different.

EXAMPLE 2 TABLE 5
Influence of dietary fish oil on alteration of number and distribution of PAS⁺ goblet cells
in descending colon crypts by CPT-11 treatment.

| | | Descending colon | | | Duodenum | | |
|---|---|---|---|---|---|---|---|
| | | Number | Mean (±SD) | Mean (±SD) | Number | Mean (±SD) | Mean (±SD) |
| Diet | | goblet | goblet | height of | goblet | goblet | height of |
| n = 5 | | cells | cells/crypt | goblet cells | cells | cells/crypt | goblet cells |
| mice/group | CPT-11 | scored | column^{a} | in crypt | scored | column^{a} | in crypt |
| Chow | − | 88 | 5.5 ± 1.8^{c} | 16.4 ± 6.2^{b} | 73 | 2.4 ± 1.3^{b} | 13.6 ± 6.0^{b} |
| 7% Corn oil | + | 107 | 8.2 ± 2.7^{b} | 13.1 ± 6.2^{c} | 30 | 1.3 ± 0.6^{c,d} | 12.1 ± 3.7^{b,c} |
| 3% Fish oil | + | 89 | 5.6 ± 1.9^{c} | 13.2 ± 6.3^{c} | 40 | 1.7 ± 1.3^{c} | 11.4 ± 4.2^{b,c} |
| 6% Fish oil | + | 78 | 5.3 ± 2.4^{c} | 11.5 ± 5.5^{c} | 24 | 1.0 ± 1.0^{d} | 10.3 ± 3.6^{c} |

^{a}Mean number of goblet cells per crypt column scored in midaxially sectioned crypts
^{bcd}Column means that share the same superscript are not significantly different

### EXAMPLE 3

Summary

The invention can be prepared as a dietary supplement either as a food additive or a separately manufactured supplement. It can be used after tumors are growing in the host. Its use should precede the beginning of therapy by at least one week, and preferably 2 weeks, then be continued for the duration of the treatment. When used as a food additive it will need to be replaced daily to avoid decomposition. When the invention was used in this manner, and as detailed in the Methods and Materials below for this example, human lung cancer cells designated A549 were implanted subcutaneously on the backs of nude mice. The tumors were allowed to grow to about 5 mm in diameter then the diet was changed to include 20% corn oil or 19% fish oil/ 1% corn oil. The mice were fed the high oil diets for ten days to allow substitution of the dietary fatty acids into cellular membranes before treatments were initiated. The treatment was doxorubicin (DOX) at 3.8 mg/kg body weight i.v. once each five days with or without ferric citrate dietary supplementation. The extra iron was added to the diet with the idea that the iron would serve as a prooxidant to add to the prooxidation potential of DOX. It was found that there was significant tumor regression in the mice which consumed fish oil diets before and during DOX treatment but not in the mice which consumed corn oil diets before and during DOX treatment. To our knowledge, this is the first observation demonstrating that dietary fish oil can increase the efficacy of a chemotherapeutic drug against a human cancer, in this case, lung cancer.

Methods and Materials

Tumor Cells

A549 human lung cancer cells (American Type Culture Collection, Rockville, Md.) were cultured for injection in nude mice. The culture medium was an enriched L15:SMEM base media supplemented with other factors as described previously [17].

Animals

Twenty five male nude mice were allowed to acclimate for one week then were inoculated with tumor cells. The mice were housed under aseptic conditions (positive air pressure in a designated nude mouse room, cages, bedding, water and food were sterilized, cages had microisolator tops) in a temperature (24° C.) and light controlled (12 h light per day) room. All mouse handling was carried out under a laminar flow hood. Our Institutional Animal Care and Use Committee approved all animal use and handling before commencing the experiment. The animal care facilities are accredited by the American Association for the Accreditation of Laboratory Animal Care. Experimental Design-Cultured A549 cells were harvested, rinsed then suspended in serum-free L15:SMEM culture medium. Cells in suspension were counted using a hemocytometer and the cell count was adjusted to 108/ml. The suspension was kept well mixed during the time of injection. A549 cells (5×10e6 cells in 0.05 ml of serum free media) were injected s.c. on the upper back of each mouse.

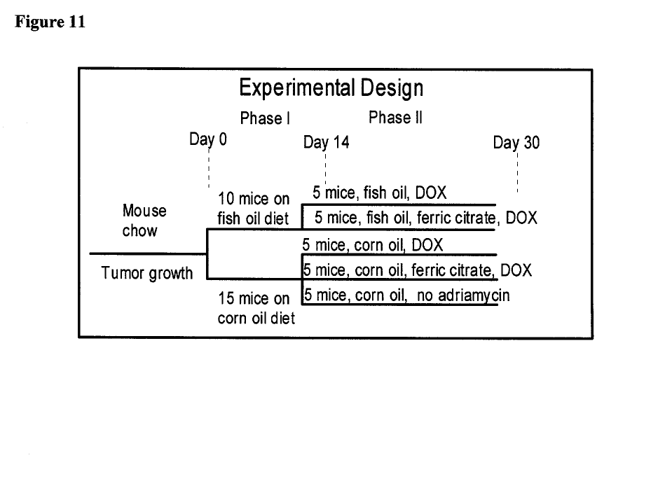

The experimental design for the tumor bearing mice is diagrammed in FIG. 11, day 0 is the day of change to the high corn oil or fish oil diets. Mice were fed a regular mouse chow diet while the tumors were allowed to grow to about 5 mm diameter. This allowed the tumors to become established as growing tumors in the host mice before onset of the experimental diets. The tumor bearing mice were then divided into groups and placed on diets based on the AIN-76A diet but modified to contain either 20% corn oil or 19% menhaden fish oil with 1% corn oil. The compositions of the experimental diets are shown in Table I. One percent corn oil was included in the fish oil diets to prevent the complications of essential fatty acid deficiency. Fifteen mice received the high corn oil diet and 10 mice received the high fish oil diet. The mice were maintained on these diets for ten days to allow substitution of cellular membrane fatty acids before beginning treatment with DOX or DOX and ferric citrate. The diets were prepared weekly, individual daily portions for each cage were packaged and the packages were stored in sealed containers at −2° C. to suppress spontaneous lipid peroxidation. The food was replaced daily to prevent consumption of oxidized lipids.

Treatment, defined as DOX alone or DOX with supplemental ferric citrate in the diet, was initiated after ten days on the corn oil or fish oil diets. Supplemental ferric citrate was added to the diet of five mice on fish oil diet and five mice on corn oil diet at a rate of 0.3% of the dry weight of the food. An untreated control group of 5 mice continued on the corn oil diet. Doxoribicin was obtained as Adriamycin PFC (Pharmacia & Upjohn, Inc. Kalamazoo, Mich.) (doxorubicin hydrocholoride for injection, USP) at a concentration of 2 mg/ml in the sterile isotonic solution. The DOX was stored under refrigeration and protected from light. A dose of 3.6 mg DOX/kg body weight (about 0.05 ml/ 28 g mouse) was injected into the lateral tail vein of the mice once each 5 days.

Tumor lengths and widths and body weights were measured three times weekly. Measurements were entered directly into an Excel spreadsheet. Tumor sizes were calculated using the formula for the volume of a prolate spheroid,

V=4/3*3.14*L/2*W/2*D/2

The width measurement was used as the depth of the tumor. This shape was a good approximation of the shape of the tumors.

The experiment was terminated 18 days after the initiation of DOX treatment. The mice were anesthetized using a ketamine/S. A. rompun mixture (0.2 cc/ 25 g weight, IM) prepared by our Laboratory Animal Resources veterinarian, then terminated by cervical dislocation. Mice in the untreated group consuming corn oil and the group consuming corn oil and treated with ferric citrate and DOX had to be killed early because of the large tumor size.

Statistical Analyses

The growth of the tumors was divided into two phases: 1) Phase I was defined as the ten days during consumption of the corn oil or fish oil diets plus four days for initiation of a response to the treatment. 2) Phase II, the final 14 days, was defined as the time of response to the treatment. Linear regression analysis was used to determine if the change in the mean tumor size during Phase I or Phase II of the experiment showed a significant linear regression and to determine the slope (rate of growth of the tumor) of each linear regression. A significant positive slope indicated tumor growth, a significant negative slope indicated tumor regression and a non-significant slope indicated no growth. Slope analysis for differences between the regression of the mean tumor volume for each group during the first or second phase of the study was performed by PRISM (GraphPad Software, San Diego, Calif.) using the general linear model procedure to generate an ANOVA. The ANOVA indicated that differences were present, thus a T-test was generated between each pair of lines against the null hypothesis that there was no difference between the slopes. A p<0.05 was used to indicate that there was a significant difference between slopes of the regression lines and that the tumor growth rates represented by the slopes were significantly different.

Results

Body Weight Change

Table II shows the mean change in body weight between day 0 (initiation of the experimental diets) and day 28 (termination) of the experiment. The results of ANOVA of the body weight showed that there were no significant differences in the mean change in body weight due to the diet or treatment of the mice. At this dose of DOX and on these diets, all groups gained weight over the course of the experiment. This indicates that the mice tolerated the diets and treatments equally well. There was no indication of diarrhea or gastric distress in any of the mice.

Tumor Growth

A graph of the mean tumor size over the time of the experiment is shown in FIG. 12. Day 0 is the day that the diets of the mice were changed to the high corn oil or high fish oil diets. The slope of the mean tumor size of each group was determined between day 0 and day 14 (Phase I) and day 14 to day 28 (Phase II). Slopes of the linear regression for each group are shown in Table III. A summary of the results of ANOVA of the tumor growth rates followed by T-tests between each pair of slopes follows:

1. There was not a significant difference in the rate of growth of the tumor due to the dietary composition during phase I of the experiment, regardless of whether the mice were consuming 20% corn oil or 19% fish oil in the diet.

2. The type of fat in the diet, however, did make a significant difference in the efficacy of DOX treatment. Specifically, DOX halted the growth of the tumors in the group of mice consuming corn oil, that is, the slope of the regression line for the growth of these tumors was not significantly different from zero (a horizontal line indicates no growth over the time of treatment). However, the tumors in the mice consuming fish oil and being treated with DOX significantly regressed, that is, the slope of the linear regression line for this group was significantly negative.

3. The type of oil in the diet made a significant difference when the treatment was a combination of supplemental iron and DOX. In the mice fed corn oil, iron counteracted the growth inhibitory effect of the DOX. In the mice fed fish oil, the tumors significantly regressed when the treatment included iron and DOX.

### Example 3

Summary

Dietary Fish Oil Sensitizes Human A549 Lung Cancer Xenografts to Doxorubicin Chemotherapy

Xenografts of the A549 human lung cancer cell line were allowed to grow in nude mice to at least 5 mm diameter then diets were changed to modified AIN-76 diets containing 19% w/w fish oil (FO) or 20% w/w corn oil (CO). Ten days later dietary ferric citrate (0.3% w/dry wt) was added and doxoribicin (DOX) treatment (3.6 mg/kg iv each 5 days for 18 days) commenced. Treatment with DOX halted the growth of tumors in the CO fed mice. However, in those mice which consumed FO or FO with ferric citrate, treatment with DOX caused significant reduction in the tumor growth rate.

Conclusions

In this example, we show that the efficacy of doxorubicin against A549 lung cancer xenografts was clearly increased when the diet was supplemented with fish oil and that there were no observed harmful side effects to the mice due to the consumption of fish oil. We do not know the mechanism of this effect, but other reports do provide clues to how fish oil works to increase the efficacy of DOX chemotherapy. For example, increased lipid peroxidation in the tumor is one likely mechanism for the increased efficacy of DOX following consumption of fish oil. One mechanism of action for DOX is the formation of DOX-metal complexes and the production of free radical complexes [Dorr & VonHoff, 1994, Cancer Chemotherapy Handbook, Appleton &Lange, Conn.]. The results of numerous reports show that membrane fatty acids of normal tissues [Borgeson et al. 1989, Lipids 24: 290-295; Lands et al., 1990, Lipids 25:505-516; Reddy & Sugie, 1988, Cancer res. 48:6642-6647; Rose et al. 1994, Nutr. Cancer 22: 131-141; Shao et al., 1995, Lipids 30: 1035-1045] and of tumors [Borgeson et al., 1989, Lipids 24: 290-295] become more unsaturated when the mice consume fatty acids from fish oil instead of corn oil. Thus, providing the PUFA substrate in the cell membranes would increase the generation of free radicals and would increase the oxidative damage from free radicals. In fact, the increased unsaturation of membrane lipids was associated with increased lipid peroxidation and decreased tumor growth in MDA-MB 231 xenografts treated with edelfosine [Hardman et al., 1997, Brit. J. Cancer 76: 347-354] or in MX-1 xenografts treated with DOX compared to mice fed corn oil [Shao et al., 1995, Lipids 30: 1035-1045].

Other mechanisms have also been proposed to account for the suppression of cancer growth by fish oil or combinations of fish oil and a drug. These mechanisms include:

1) Decreased activity of PGE2 following dietary fish oil [Hornstra et al, 1990, Adv. Prostaglandin Thromboxane Leukot. Res. 21: 225-228; Weber & Sellmayer, 1990, Adv. Prostaglandin Thromboxane Leukot. Res. 21: 217-224]. Decreased PGE2 is associated with increased immune activity [Hwang, 1989, FASEB J. 3: 2052-2055] and decreased tumor promotion and growth [deVries & Van Noorden, 1992, Anticancer Res. 12:1513-1522].

2) Decreased activity of protein kinase C [PKC], which has been associated with reversal of drug resistance [Das et al., 1998, Prostaglandins Leukot Essent Fatty Acids 58: 39-54] and slowed angiogenesis (reviewed in [McCarty, 1996, Med Hypoth 46:107-115].

In addition, the n-3 fatty acids of fish oil have been shown to be beneficial to the patient by suppressing cancer cachexia [Beck et al., Canc. Res. 51: 6089-6093; Price & Tisdale, 1998, Cancer Res. 58: 4827-4831; Tisdale, 1993, Prostaglandins Leukot Essent Fatty Acids 48: 105-109] and by improving the response to radiotherapy [Das et al., 1998, Prostaglandins Leukot Essent Fatty Acids 58: 39-54]. Use of the n-3 fatty acids derived from fish oil as an adjuvant to therapy has the potential to increase the efficacy of the chemo- or X-radiation therapies in current use. Our future studies to investigate the mechanisms of n-3 fatty acids to increase the efficacy of cancer therapy will allow us to devise even more effective cancer treatment strategies.

TABLE I
Composition of the diet by weight percent (g/100 g of food)

| Ingredient | 20% Corn oil | 19% Menhaden oil |
|---|---|---|
| | | 1% Corn oil |
| Corn oil | 20.0 | 1.0 |
| Menhaden oil | | 19.0 |
| Sugar | 27.9 | 27.9 |
| Casein | 23.2 | 23.2 |
| Cornstarch | 17.4 | 17.4 |
| AIN-76 vitamin mixa | 1.15 | 1.15 |
| AIN-76 mineral mixa | 4.06 | 4.06 |
| Choline bitartrate | 0.23 | 0.23 |
| DL-methionine | 0.35 | 0.35 |
| Cellulose | 5.8 | 5.8 |
| Total | 100.1 | 100.1 |
| Composition of the diets by | | |
| % caloriesb | | |
| Protein | 20.6 | 20.6 |
| Carbohydratec | 40.1 | 40.1 |
| Fat | 39.3 | 39.3 |
| Energy content of each diet | 4.52 | 4.52 |
| kcal/g | | |

a α-tocopherol is 0.02 g/100 g and ferric citrate (16-17% Fe+++) is 0.02 g/100 g of the basal diet.

b Caloric content is calculated at 4 kcal/g for protein and carbohydrate and 9 kcal/g for fat. The diet which included a prooxidant (iron) had 0.3 g/100 g of ferric citrate (16-17% Fe+++) added to the 19% MO or 20% CO diet.

c The % of calories from carbohydrate include the calories from sucrose, cornstarch and sucrose in the vitamin and mineral mix.

Diet Components and Chemicals

Purified high nitrogen casein, pure corn starch, Alphacel (non-nutritive bulk cellulose) AIN-76 vitamin mixture, AIN-76 mineral mixture and choline bitartrate (99% pure) was obtained from ICN Nutritional Biochemicals, Cleveland, Ohio. Imperial brand (Sugarland, Tex.) extra fine pure cane sugar and 100% pure corn oil (Wesson) was purchased locally. D.L. methionine (cell culture, M. W. 149.2), menhaden fish oil and ferric citrate was purchased from Sigma, St. Louis, Mo.

TABLE II
The mean change in body weights of the groups of mice from day 0 to
day 28 of the experiment (the time of consumption of the corn oil or fish
oil diets).

| Final diet group (n = 4 or 5) | mean (g) + SD* |
|---|---|
| Corn oil | +5.6 + 2.0 |
| Fish oil | +4.8 + 2.6 |
| Cornoil + Fe | +4.8 + 1.6 |
| Fish oil + Fe | +4.2 + 1.2 |
| Corn oil no DOX | +3.3 + 0.7 |

*ANOVA showed that there were no significant differences in the change in body weight due to the diet or treatment of the mice.

TABLE III
Growth rate of A549 human lung tumors (mean mm3 per day + SD of
slope).

| Final diet/treatment group | Phase Ia | Phase II |
|---|---|---|
| Corn oil; DOX | 14.8 + 1.9 | −1.5 + 1.8b |
| Fish oil, DOX n | 16.2 + 1.8 | −11.1 + 1.5c |
| Corn oil + iron, DOX | 15.9 + 1.3 | 34.1 + 4.2d |
| Fish oil + iron, DOX | 11.2 + 2.3 | −13.1 + 4.2c |
| Corn oil no DOX | 14.9 + 2.0 | 14.9 + 2.0 |

a Linear regression analyses showed that during phase I, all slopes were significantly different from 0. ANOVA of the slopes showed that the growth rates of the tumors (slopes) were not significantly different from each other during Phase I when mice were consuming either a corn oil or a fish oil diet without any DOX treatment.

b, c, d Linear regression analyses showed that the tumor growth rate (slope of the regression line) of the group of mice which consumed corn oil and was treated with DOX was not significantly different from a slope of 0. The tumor growth rate of all other groups was a significant positive or negative slope. ANOVA of the slopes showed that growth rates (slopes) with the same letter are not significantly different, growth rates with different letters are significantly different.

The industrial applicability of this invention is in the area of human and animal health, in particular cancer treatment. It also has potential utility in other diseases where inflammation is a contributing factor to disease. The commercialization of this invention is intended to lead to improved outcomes in patients with cancer and to enhance their quality of life during cancer therapy by increasing the health of normal cells and tissues. It will be intended as an adjuvant to, and used with, currently accepted standard treatments for cancer and other diseases. In particular, the invention will have use where the treatment may damage the healthy tissues and thus cause morbidity for the patient, or may reduce the patient's therapeutic response due to non-compliance or discontinuation of the treatment. Examples include sores in the mouth or other locations in the gastrointestinal tract, and effects on the renewal of normal bone marrow and blood cell formation. Thus, the invention may have a secondary effect of increasing therapeutic efficacy of the proposed treatment regimen, directly ort indirectly. The overall outcome for the use of this invention will be the saving of lives or increasing lifespan and productivity, increased quality of life for patients receiving treatment, and the resultant economic impact engendered by those results.

### Drawings and Figures

### EXAMPLE 1

Toxicity of and Moderating Side Effects of CPT-11 Treatment by the Invention, Accelerated Action Fatty Acid (AAFA™), a Concentrated Omega 3 Fatty Acid Fish Oil Formulation

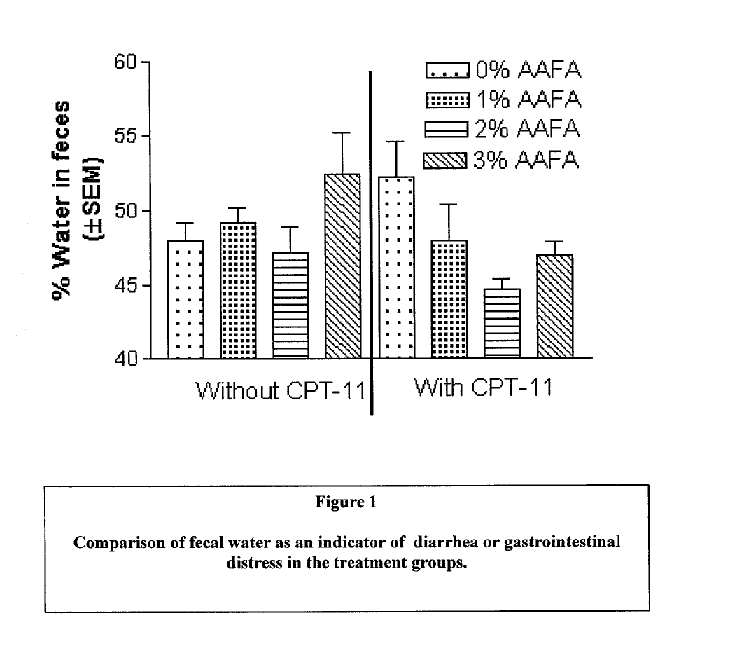

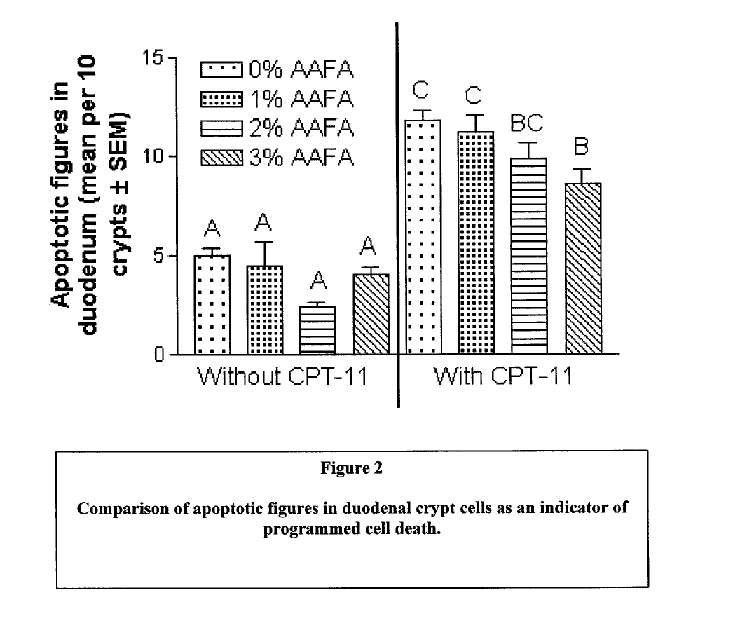

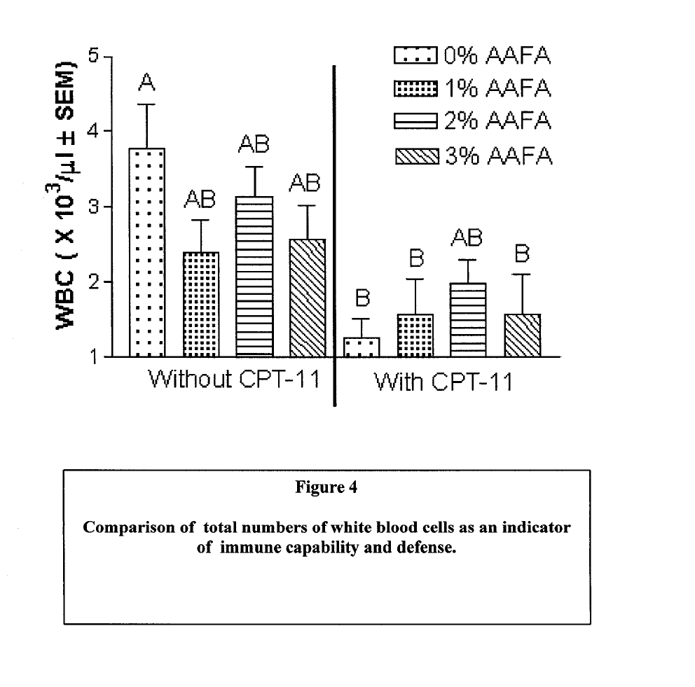

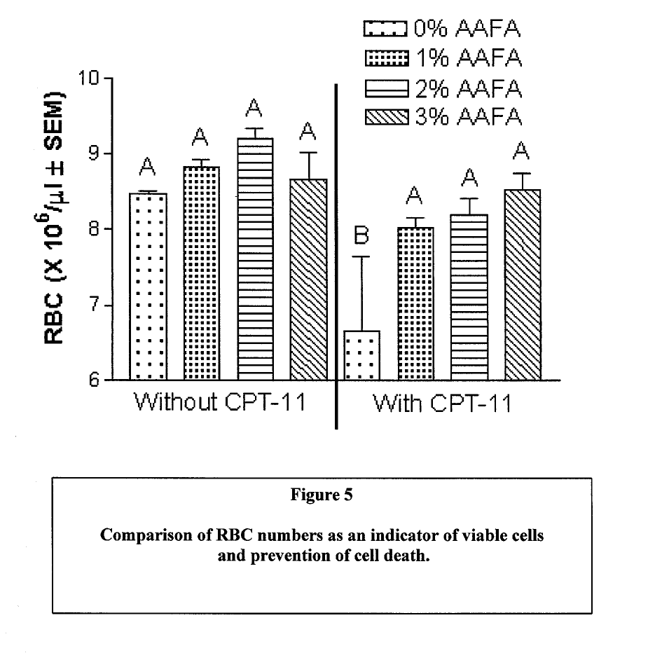

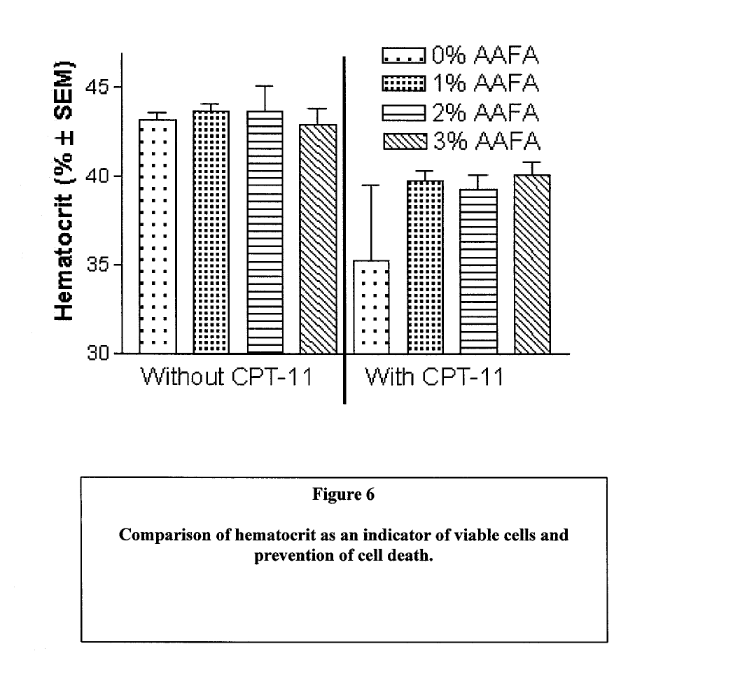

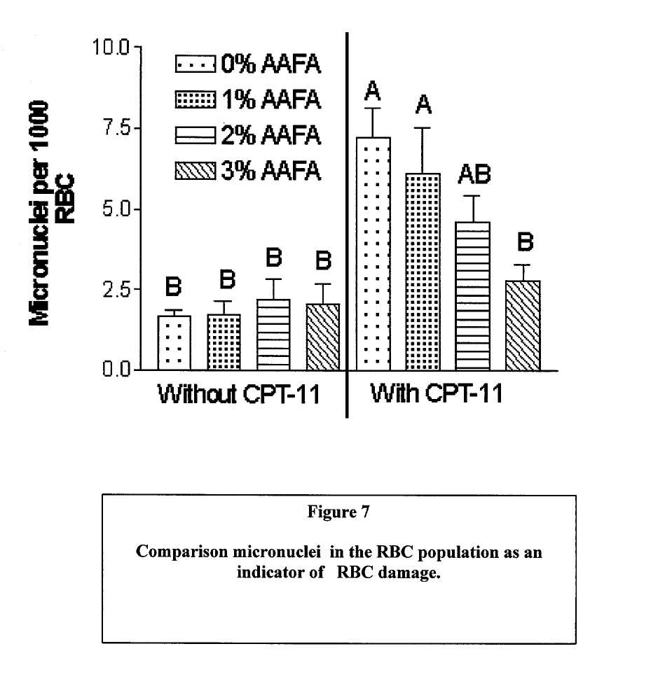

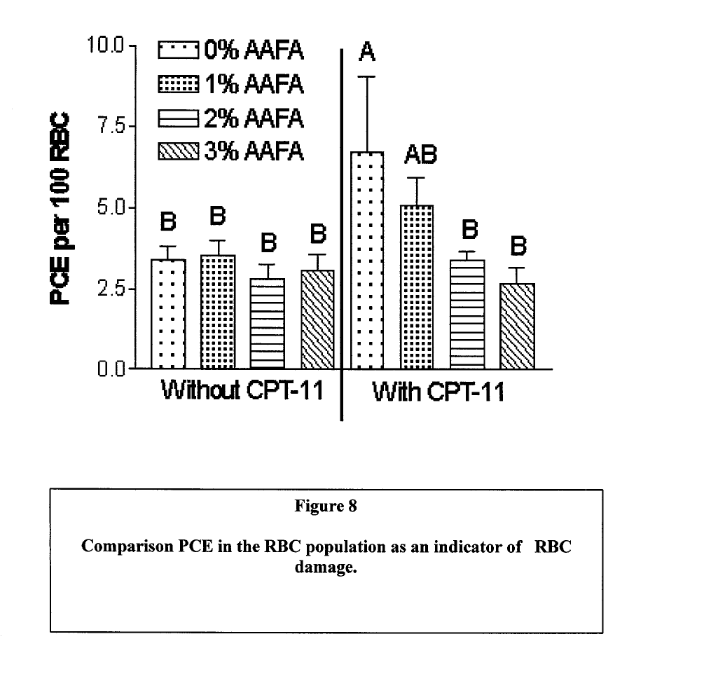

Legend to FIGS. 1-8

A series of graphs were prepared to describe the observations of this study series. Results of one-way analysis of variance of the data followed by Student-Newman-Keuls multiple range tests as appropriate. Data is presented graphically, the Y-axis of each graph is labeled to identify each set of data. On each graph, groups that do not share a superscript are significantly different (p<0.05).

1. A formulation and methods for using fatty acids, said fatty acids derived from concentrated fish oil and being comprised of .omega.3 fatty acids of at least 50% of the total volume, with EPA (20:5) and DHA (22:6) at minimum limits of 30% and 10% for a total EPA+DHA component of at least 40%.

2. The formulation and method of claim 1 wherein said fish oils are selected from a group consisting of menhaden oil, salmon oil, anchovy oil, herring oil, and mixtures thereof; and wherein values of potentially harmful pesticides and heavy metals do not exceed 2 or 5 ppm, respectively.

3. The formulation and method of claim 1 wherein natural oils containing .omega.3 fatty acids are derived from plant oils, marine plankton oils, or fungal oils.

4. The formulation and method of claims 1, 2 and 3, wherein said fish oils or natural oils may or may not contain antioxidants such as α-tocopherol or retinoids as preservatives or additives, and may be a component of a formulated nutritional supplement product.

5. A method of reducing the side effects of chemotherapy or radiation therapy for multiple types of cancer and other diseases by administering a diet supplemented with 2% to 6% by weight of oil enriched for .omega.3 fatty acids in a natural oil composition as noted in claims 1-4; whereby said method augments the health of normal cells and tissues, and improves general patient health, including diminishment of cachexia or other side effects that can include gastrointestinal effects, myelosuppression, cachexia, inhibition of immune function, increased susceptibility to infection, organ toxicity, and changes in blood vessel growth or architecture.

6. The method of claims 1-5 wherein normal cell differentiation and function are promoted, and abnormally induced or accelerated cell death by apoptosis or other mechanisms are inhibited, as exemplified in studies of the gastrointestinal tract but applicable to other organs and tissues, including the cardiovascular system.

7. The anticancer chemotherapy agent preferably is selected from alkylating agents, antimetabolites, natural products, hormones and antagonists, and miscellaneous agents, such as radiosensitizers; wherein the anticancer agents useful in the practice of this invention are known compounds and/or can be prepared by techniques known in the art.

8. The anticancer agent can be used alone or in combination with one or more anticancer agents, and treatment with the oil enriched for .omega. 3 fatty acids in a natural oil composition as noted in claims 1-4 preferentially begins 5-14 days prior to chemotherapy, but can be delivered also during therapy, and post-therapy, with expected benefit.

9. The method for claims 1-8 for side effects due to treatment with chemotherapy in families of compounds that inhibit cell division by a variety of mechanisms, including direct effects on the deoxyribonucleic acid (DNA) molecule, DNA binding molecules, or structural molecules such as microtubules and microfilaments.

10. The method for claims 1-8 for side effects due to treatment with chemotherapy in families of compounds that augment cell necrosis or apoptosis, which is programmed cell death, or in families of compounds that alter membrane transport, receptors, signaling, or function.

11. A method of augmenting immunotherapy for cancer and other diseases by administering a diet supplemented with 2% to 6% by weight of formulations as defined in claims 1-4, wherein said oil is enriched for omega.3 fatty acids in a natural oil composition; whereby said method augments the health of normal cells and tissues, and which preferentially begins 7-14 days prior to chemotherapy, but can be delivered also during and post-therapy with expected benefit.

12. The method of claims 1-11 wherein the dietary supplement is administered enterally alone, in capsules or associated with a carrier; or is administered parenterally alone, or associated with a carrier.

13. The formulation and method for claims 1-12 wherein the supplement is prepared with flavorings or other enhancers added at <5% of the final composition, or is prepared as a synthetic mixture.

14. The formulation and method of claims 1-12 wherein the dietary supplement augments therapy against cancer and other diseases in all stages of treatment including stages where tumors that have become resistant to therapy, or where the dietary supplement is prepared with other nutritional supplements or foods, or beneficial additives to augment the cancer therapy effects.

15. The formulation and method of claims 1-12 wherein the dietary supplement is used as an adjuvant with other therapies, that may include surgery, radiation, molecular-based, genetic, receptors, enzymes, or antibody therapies; but where preventive or maintenance amounts mat be lessened to a concentration of 1-2% of the diet for general health.

16. The formulation and method of claims 1-15 wherein the dietary supplement is used to treat or minimize liver damage or enlargement due to chemotherapy or other factors, including infectious agents such as hepatitis viruses; but where preventive or maintenance amounts mat be lessened to a concentration of 1-2% of the diet for general health benefits.

17. The formulation and method of claims 1-15 wherein the dietary supplement is used to enhance growth and differentiation of cardiovascular and blood cells, including white blood cells and their immune function; red blood cell growth and differentiation, for example, as a means to diminish anemia; and for cardiovascular health; but where preventive or maintenance amounts mat be lessened to a concentration of 1-2% of the diet for general health benefits.

18. The formulation and method of claims 1-15 wherein the dietary supplement is used to augment the general health of the organ systems, tissues and cells in the immunocompromised patient, whether acquired by drug treatment, radiation treatment, infection, transplantation, or genetic constitution; but where preventive or maintenance amounts mat be lessened to a concentration of 1-2% of the diet for general health benefits. This would include acquired immune deficiency syndrome.

19. The formulation and method of claims 1-15 wherein the dietary supplement is used to augment the general health of the organ systems, tissues and cells in the patient with arthritis or other bone and joint diseases where reduction of inflammation may bring relief; but where preventive or maintenance amounts mat be lessened to a concentration of 1-2% of the diet for general health benefits. This would include autoimmune diseases.

20. The formulation and method of claims 1-4 wherein the dietary supplement is used to augment the general health of the organ systems, tissues and cells in the neurally compromised or disabled patient, whether acquired by drug treatment, radiation treatment, infection, transplantation, or genetic constitution so that they exhibit more normal behavior either due to reduced pain or enhancement of positive neural regulatory mechanisms; but where preventive or maintenance amounts mat be lessened to a concentration of 1-2% of the diet for general health benefits.
